(19) United States
(12) Patent Application Publication
(10) Pub. No.: US 2019/0287646 A1
(43) Pub. Date: Sep. 19, 2019

(54) IDENTIFYING COPY NUMBER ABERRATIONS

(71) Applicant: GRAIL, Inc., Menlo Park, CA (US)

(72) Inventor: Earl Hubbell, Palo Alto, CA (US)

(21) Appl. No.: 16/352,214

(22) Filed: Mar. 13, 2019

Related U.S. Application Data
(60) Provisional application No. 62/642,507, filed on Mar. 13, 2018.

(51) Int. Cl. G16B 20/10; G16B 40/00; G16B 30/10; C12Q 1/68

(52) U.S. Cl. CPC G16B 20/10; C12Q 1/68; G16B 30/10; G16B 40/00

(57) ABSTRACT

A system can identify a source of a copy number change in a sample based on a comparison of properties of the sample to a second sample. Sequence reads categorized in bins of a genome are obtained from a first sample and a second sample. A determination is made whether each bin categorized by the sequence reads is statistically significant based on, for example, a bin sequence read count, an expected sequence read count, and a yin variance estimate for the bin. Likewise, a determination is made whether, for the first sample and the second sample, each segment of the genome is statistically significant based on a segment sequence read count and a segment variance estimate. Statistically significant bins and segments of the first sample are compared to statistically significant bins and segments of the second sample, and a copy number change source is identified based on the comparison.

### CROSS REFERENCE TO RELATED APPLICATIONS

This application claims the benefit of and priority to U.S. Provisional Application No. 62/642,507, filed on Mar. 13, 2018, which is incorporated herein by reference in its entirety for all purposes.

### BACKGROUND

This disclosure generally relates to detecting copy number changes in a genome, and more specifically to detecting copy number aberrations that are likely due to the presence of solid tumor tissue.

Copy number aberrations (CNAs), which are changes in copy number in somatic tumor tissue, play an important role in the etiology of many diseases such as cancers. CNAs include, for example, amplification(s) and deletion(s) of genomic regions. Recent advances in sequencing technologies have enabled the characterization of a variety of genomic features, including CNAs. This has led to the development of bioinformatics approaches to detect CNAs from next-generation sequencing (NGS) data.

However, accurate identification of CNAs in the genome of an individual can be confounded by other changes that are present in an individual. For example, other copy number variations (CNVs), such as copy number changes in non-tumor cells, which may not be indicative of a disease, can often be incorrectly identified as a CNA associated with disease. There is a need for methods of accurately identifying CNAs that derive from a somatic tumor source while removing confounding factors, such as the presence of CNVs that originate from a non-tumor source.

### SUMMARY

Embodiments described herein relate to methods of identifying a source of a copy number event detected in sequence reads derived from cell free DNA. A source of a copy number event can be one of a germline source (e.g., a copy number variation present in germline cells), a somatic non-tumor source (e.g., a copy number variation derived from cells of a blood cell lineage), or a somatic tumor source (e.g., a copy number aberration derived from solid tumor cells). By identifying a source of a copy number event, non-tumor related copy number events can be filtered out and removed. This increases the specificity of a copy number aberration caller and can be beneficial for applications such as early detection of cancer.

Cell-free DNA (cfDNA) and genomic DNA (gDNA) are extracted from a test sample and sequenced (e.g., using whole exome or whole genome sequencing) to obtain sequence reads. cfDNA sequence reads and gDNA sequence reads are separately analyzed to identify the possible presence of one or more copy number events in each respective sample. Here, the source of copy number events derived from cfDNA can be any one of a germline source, somatic non-tumor source, or somatic tumor source. The source of copy number events derived from gDNA can be either a germline source or a somatic non-tumor source. Therefore, copy number events detected in cfDNA but not detected in gDNA can be readily attributed to a somatic tumor source.

Embodiments of the described method include performing a bin-level analysis across bins of a genome (e.g., bins are on the order of 50 to 1000 kilobases). For each sample, sequence read counts are categorized into individual bins across the genome. The total sequence read count in each bin is normalized to account for non-biological biases that may arise due to processing conditions. These non-biological biases may include processing biases (e.g., guanine cytosine content bias and mappability bias), expected sequence read counts for a bin (e.g., some bins may naturally result in higher sequence read counts than others), expected variance for a bin (e.g., some bins may be noisier than other bins), and variance of the sample (e.g., some samples may be noisier than other samples). By normalizing the sequence read counts of bins to account for non-biological biases, bins whose normalized sequence read counts differ from expected are indicative of a copy number event. Such bins are referred to hereafter as statistically significant bins.

Embodiments of the described method further include performing a segment-level analysis of segments in the genome. Each segment includes one or more bins across the genome and is generated such that segments adjacent to one another have segment sequence read counts that are significantly different from each other. The segment sequence read counts for each segment are normalized to account for non-biological biases and therefore, segments that have normalized sequence read counts that differ from expected are indicative of a copy number event. Such segments are referred to hereafter as statistically significant segments.

Statistically significant bins and statistically significant segments identified from the cfDNA sample are compared to the corresponding bins and segments in the gDNA sample. This comparison enables the identification of a source of copy number events that are indicated by the statistically significant bins and statistically segments identified from the cfDNA sample. Specifically, if a statistically significant bin or segment of the cfDNA sample is correspondingly also a statistically significant bin or segment of the gDNA sample, the copy number event is likely a copy number variation derived from a non-tumor source. In other words, either a germline event or a somatic non-tumor event likely caused the copy number event that is observed in both the cfDNA and gDNA sample. Conversely, if a statistically significant bin or segment from the cfDNA sample does not correspond to a statistically significant bin or segment from the gDNA sample, the copy number event is likely a copy number aberration. In other words, a somatic tumor event likely caused the copy number event that is observed in the cfDNA sample but not in the gDNA sample.

By identifying the source of a copy number event, copy number variations can be filtered out whereas copy number aberrations can be kept and further analyzed. Thus, the identified copy number aberrations can be further analyzed for applications such as early detection of cancer.

### BRIEF DESCRIPTION OF THE DRAWINGS

FIG. 1 is an example flow process for processing a test sample obtained from an individual to identify a copy number aberration, in accordance with an embodiment.

FIG. 2A is an example flow process for identifying a source of a copy number event identified in a cfDNA sample, in accordance with an embodiment.

FIG. 2B is an example flow process that describes the analysis for identifying statistically significant bins and segments derived from cfDNA and gDNA samples, in accordance with an embodiment.

FIG. 2C depicts an example database that stores characteristics that are used to identify a source of a copy number event, in accordance with an embodiment.

FIG. 3A is an example depiction of sequence reads in relation to bins of a reference genome, in accordance with an embodiment.

FIG. 3B is an example chart depicting expected and observed sequence read counts across different bins of a genome, in accordance with an embodiment.

FIG. 4A and FIG. 4B depicts bin scores across bins of a genome for a cfDNA sample and a gDNA sample, respectively, that are obtained from a breast cancer subject.

FIG. 5 is a graph depicting the distribution of bin scores for the gDNA sample shown in FIG. 4B in relation to corresponding bin scores for the cfDNA sample shown in FIG. 4A.

FIG. 6A and FIG. 6B depicts bin scores across bins of a genome determined from a cfDNA sample and a gDNA sample, respectively, that are obtained from a non-cancer individual.

FIG. 7 is a graph depicting the distribution of bin scores for the gDNA sample shown in FIG. 6B in relation to corresponding bin scores for the cfDNA sample shown in FIG. 6A.

FIG. 8A and FIG. 8B depicts bin scores across bins of a genome determined from a cfDNA sample and a gDNA sample, respectively, that are obtained from a non-cancer individual.

FIG. 9 is a graph depicting the distribution of bin scores for the gDNA sample shown in FIG. 8B in relation to corresponding bin scores for the cfDNA sample shown in FIG. 8A.

### DETAILED DESCRIPTION

The figures and the following description relate to preferred embodiments by way of illustration only. It should be noted that from the following discussion, alternative embodiments of the structures and methods disclosed herein will be readily recognized as viable alternatives that may be employed without departing from the principles of what is claimed.

Reference will now be made in detail to several embodiments, examples of which are illustrated in the accompanying figures. It is noted that wherever practicable similar or like reference numbers may be used in the figures and may indicate similar or like functionality. For example, a letter after a reference numeral, such as “bin 320A,” indicates that the text refers specifically to the element having that particular reference numeral. A reference numeral in the text without a following letter, such as “bin 320,” refers to any or all of the elements in the figures bearing that reference numeral (e.g. “bin 320” in the text refers to reference numerals “bin 320A” and/or “ bin 320B” in the figures).

The term “individual” refers to a human individual. The term “healthy individual” refers to an individual presumed to not have a cancer or disease. The term “ cancer subject” refers to an individual who is known to have, or potentially has, a cancer or disease.

The term “sequence reads” refers to nucleotide sequences read from a sample obtained from an individual. Sequence reads can be obtained through various methods known in the art.

The term “cell free nucleic acid,” “cell free DNA,” or “cfDNA” refers to nucleic acid fragments that circulate in an individual's body (e.g., bloodstream) and originate from one or more healthy cells and/or from one or more cancer cells.

The term “genomic nucleic acid,” “genomic DNA,” or “gDNA” refers to nucleic acid including chromosomal DNA that originates from one or more healthy (e.g., non-tumor) cells. In various embodiments, gDNA can be extracted from a cell derived from a blood cell lineage, such as a white blood cell.

The term “copy number aberrations” or “CNAs” refers to changes in copy number in somatic tumor cells. For example, CNAs can refer to copy number changes in a solid tumor.

The term “copy number variations” or “CNVs” refers to changes in copy number changes that derive from germline cells or from somatic copy number changes in non-tumor cells. For example, CNVs can refer to copy number changes in white blood cells that can arise due to clonal hematopoiesis.

The term “copy number event” refers to one or both of a copy number aberration and a copy number variation.

### Methods for Identifying a Source of Copy Number Aberrations

General Processing Steps for Generating Sequence Reads from Samples

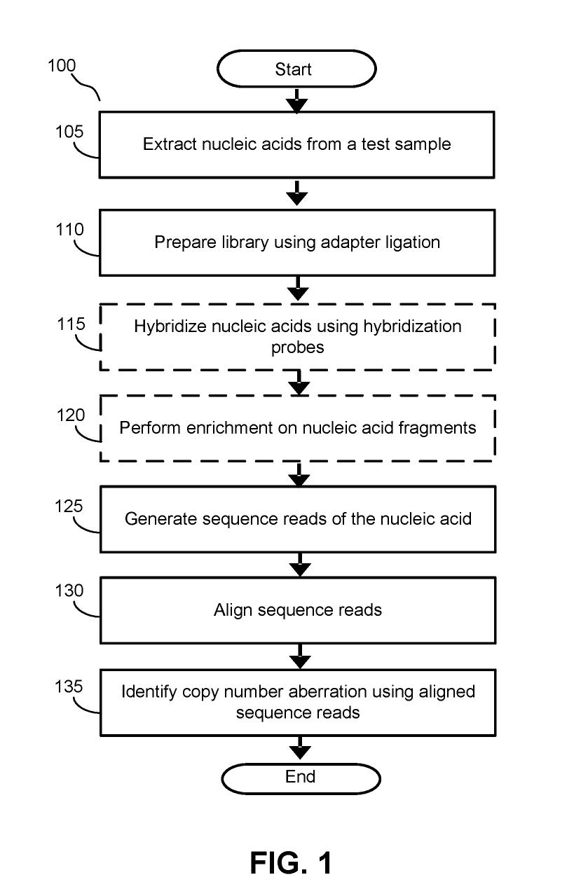

FIG. 1 is an example flow process 100 for processing a test sample obtained from an individual to identify a copy number aberration, in accordance with an embodiment. At step 105, nucleic acids are extracted from a test sample. In one embodiment, the test sample may be from a cancer subject known to have or suspected of having cancer. The test sample may be a sample selected from the group consisting of blood, plasma, serum, urine, fecal, and saliva samples. Alternatively, the test sample may comprise a sample selected from the group consisting of whole blood, a blood fraction, a tissue biopsy, pleural fluid, pericardial fluid, cerebral spinal fluid, and peritoneal fluid. In accordance with some embodiments, the test sample comprises cell-free nucleic acids (e.g., cell-free DNA). In some embodiments, the cell-free nucleic acids in the test sample originate from one or more healthy cells and from one or more cancer cells. In accordance with some embodiments, the test sample comprises genomic DNA (e.g., gDNA), wherein the gDNA in the test sample includes chromosomal DNA obtained from one or more healthy cells. In some embodiments, the one or more healthy cells are from a healthy cell, e.g., a blood lineage. For example, the one or more healthy cells can be white blood cells.

In various embodiments, the test sample includes both cfDNA and gDNA and therefore, the test sample is processed to extract both cfDNA and gDNA. In general, any known method in the art can be used for extracting DNA. For example, nucleic acids can be extracted and purified using one or more known commercially available protocols or kits, such as the QIAAMP circulating nucleic acid kit (Qiagen). In other embodiments, nucleic acids can be isolated by pelleting and/or precipitating the nucleic acids in a tube. In some embodiments, a test sample is processed to obtain a cfDNA sample and a gDNA sample from which cfDNA and gDNA can be respectively extracted. For example, a test sample can be centrifuged to separate a supernatant fluid and pelleted cells. The supernatant fluid can represent a cfDNA sample whereas the pelleted cells can represent a gDNA sample. In some embodiments, the nucleic acids in the test sample can be fragmented, for example, genomic DNA (gDNA) in a sample can be fragmented (e.g., a sheared gDNA sample) before subsequent processing.

Following extraction of nucleic acids, one of various sequencing processes can be performed. For example, the extracted nucleic acids can be used to perform one of a targeted sequencing (e.g., a targeted gene panel sequencing), whole exome sequencing, whole genome sequencing, or methylation-aware sequencing (e.g., whole genome bisulfite sequencing).

At step 110, a sequencing library is prepared. During library preparation adapters, for example, include one or more sequencing oligonucleotides for use in subsequent cluster generation and/or sequencing (e.g., known P5 and P7 sequences for used in sequencing by synthesis (SBS) (Illumina, San Diego, Calif.)) are ligated to the ends of the nucleic acid fragments through adapter ligation. In one embodiment, unique molecular identifiers (UMI) are added to the extracted nucleic acids during adapter ligation. The UMIs are short nucleic acid sequences (e.g., 4-10 base pairs) that are added to ends of nucleic acids during adapter ligation. In some embodiments, UMIs are degenerate base pairs that serve as a unique tag that can be used to identify sequence reads obtained from nucleic acids. As described later, the UMIs can be further replicated along with the attached nucleic acids during amplification, which provides a way to identify sequence reads that originate from the same original nucleic acid segment in downstream analysis.

Referring briefly to FIG. 1, steps 115, and 120 are optionally performed. For example, steps 115 and 120 are performed for targeted gene panel sequencing and whole exome sequencing. However, for whole genome sequencing, steps 115, and 120 need not be performed.

At step 115, hybridization probes are used to enrich a sequencing library for a selected set of nucleic acids. Hybridization probes can be designed to target and hybridize with targeted nucleic acid sequences to pull down and enrich targeted nucleic acid fragments that may be informative for the presence or absence of cancer (or disease), cancer status, or a cancer classification (e.g., cancer type or tissue of origin). In accordance with this step, a plurality of hybridization pull down probes can be used for a given target sequence or gene. The probes can range in length from about 40 to about 160 base pairs (bp), from about 60 to about 120 bp, or from about 70 bp to about 100 bp. In one embodiment, the probes cover overlapping portions of the target region or gene. For targeted gene panel sequencing, the hybridization probes are designed to target and pull down nucleic acid fragments that derive from specific gene sequences that are included in the gene panel. For whole exome sequencing, the hybridization probes are designed to target and pull down nucleic acid fragments that derive from exon sequences in a reference genome.

At step 120, the probe-nucleic acid complexes are enriched. For example, as is well known in the art, a biotin moiety can be added to the 5′-end of the probes (i.e., biotinylated) to facilitate pulling down of target probe-nucleic acids complexes using a streptavidin-coated surface (e.g., streptavidin-coated beads). Optionally, a second device, such as a polymerase chain reaction (PCR) device, can be used for amplification of the targeted nucleic acids.

At step 125, the nucleic acids are sequenced to generate sequence reads. Sequence reads may be acquired by known means in the art. For example, a number of techniques and platforms obtain sequence reads directly from millions of individual nucleic acid (e.g., DNA such as cfDNA or gDNA) molecules in parallel. Such techniques can be suitable for performing any of targeted sequencing (e.g., targeted gene panel sequencing), whole exome sequencing, whole genome sequencing, and methylation-aware sequencing (e.g., whole genome bisulfite sequencing).

In one embodiment, sequence reads from the sequencing library can be acquired using next generation sequencing (NGS). Next-generation sequencing methods include, for example, sequencing by synthesis technology (Illumina), pyrosequencing (454), ion semiconductor technology (Ion Torrent sequencing), single-molecule real-time sequencing (Pacific Biosciences), sequencing by ligation (SOLiD sequencing), and nanopore sequencing (Oxford Nanopore Technologies). In some embodiments, sequencing is massively parallel sequencing using sequencing-by-synthesis with reversible dye terminators. In other embodiments, sequencing is sequencing-by-ligation. In other embodiments, sequencing is single molecule sequencing. In other embodiments, sequencing is paired-end sequencing.

At step 130, sequence reads are aligned to a reference genome. In general, any known method in the art can be used for aligning the sequence reads to a reference genome. For example, the nucleotide bases of a sequence read are aligned with nucleotide bases in the reference genome to determine alignment position information for the sequence read. Alignment position information can include a beginning position and an end position of a region in the reference genome that corresponds to the beginning nucleotide base and end nucleotide base of the sequence read. Alignment position information may also include sequence read length, which can be determined from the beginning position and end position. In various embodiments, a BAM file of aligned sequencing reads for regions of the genome is obtained and utilized for analysis in step 135.

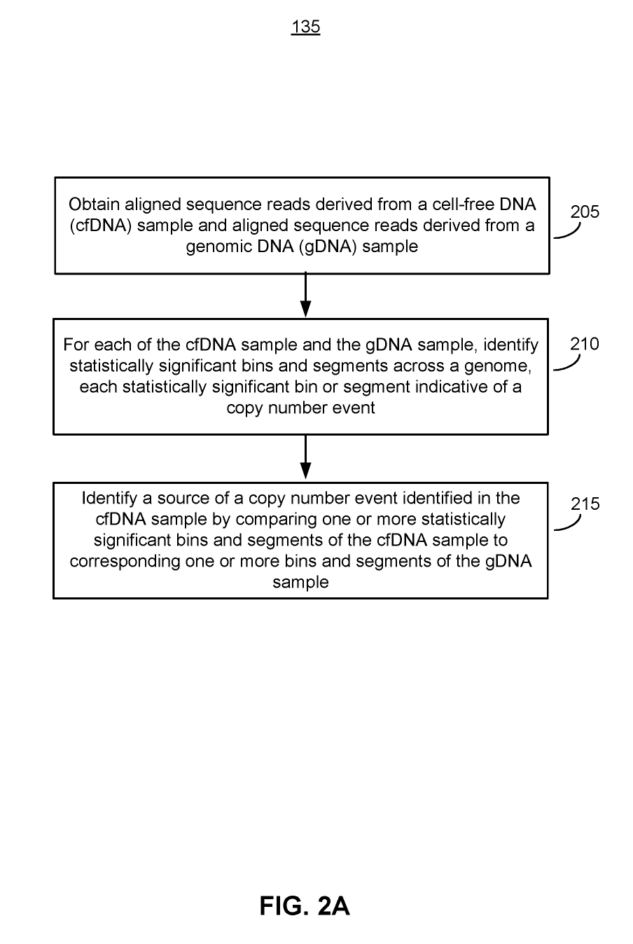

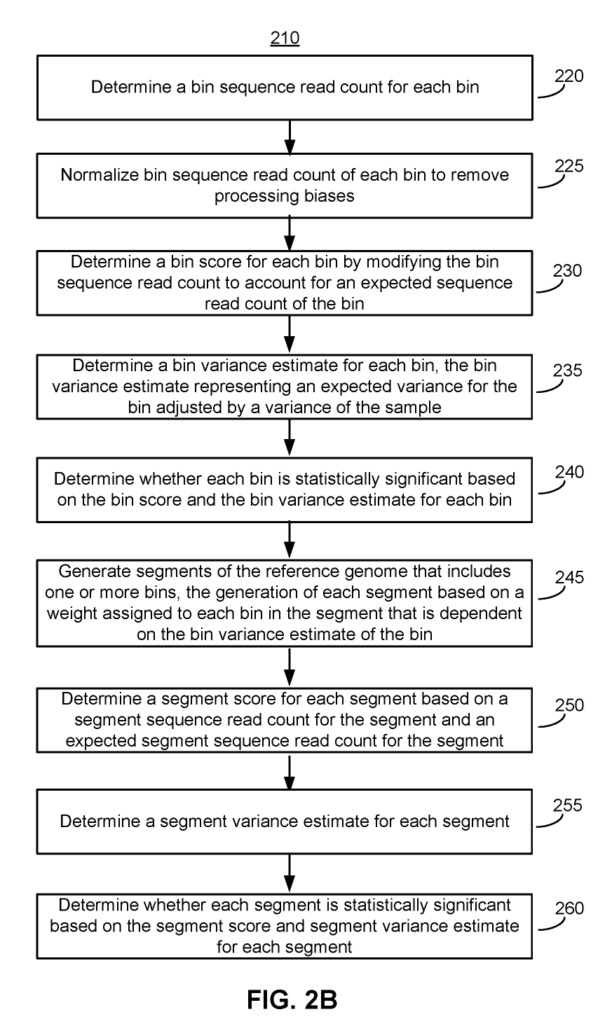

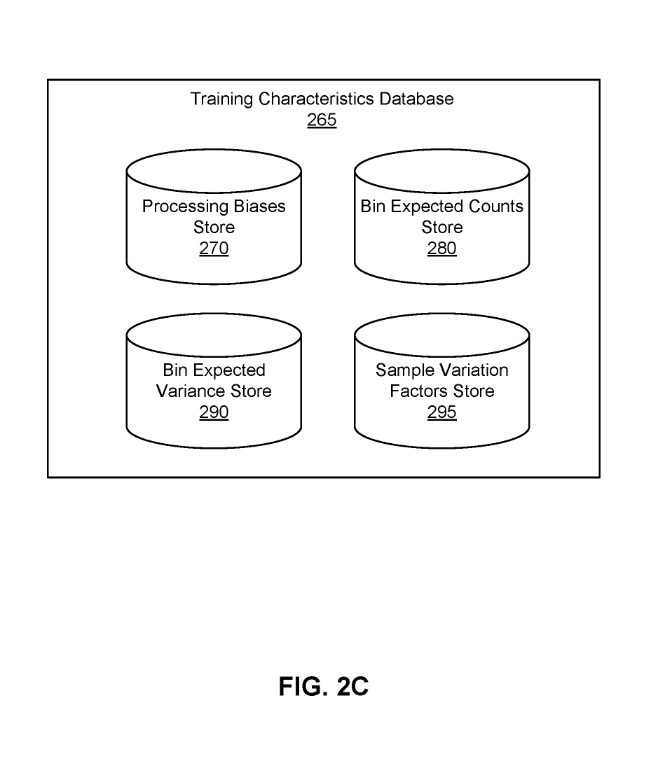

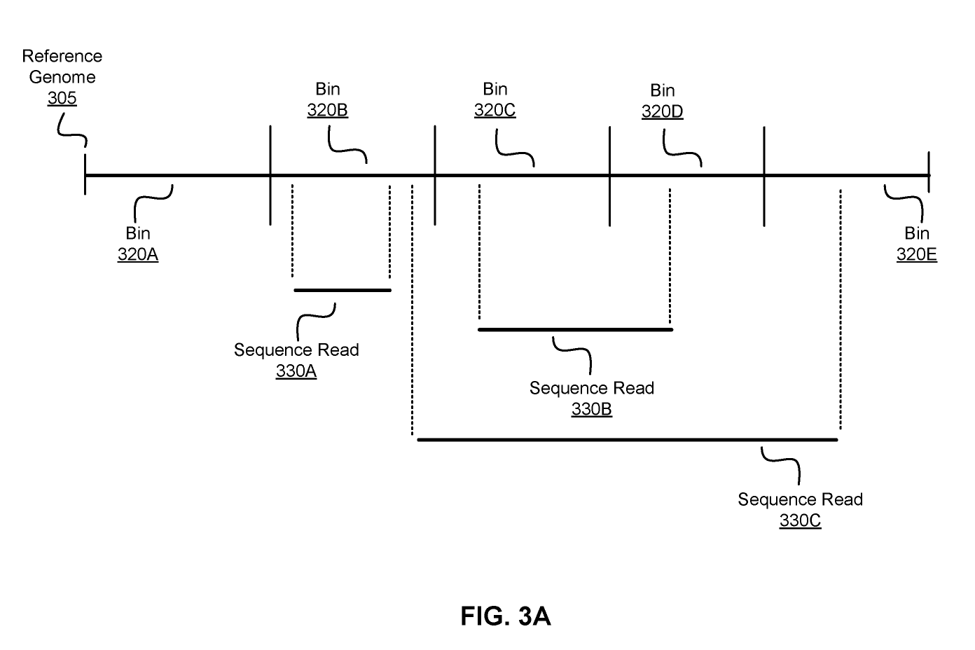

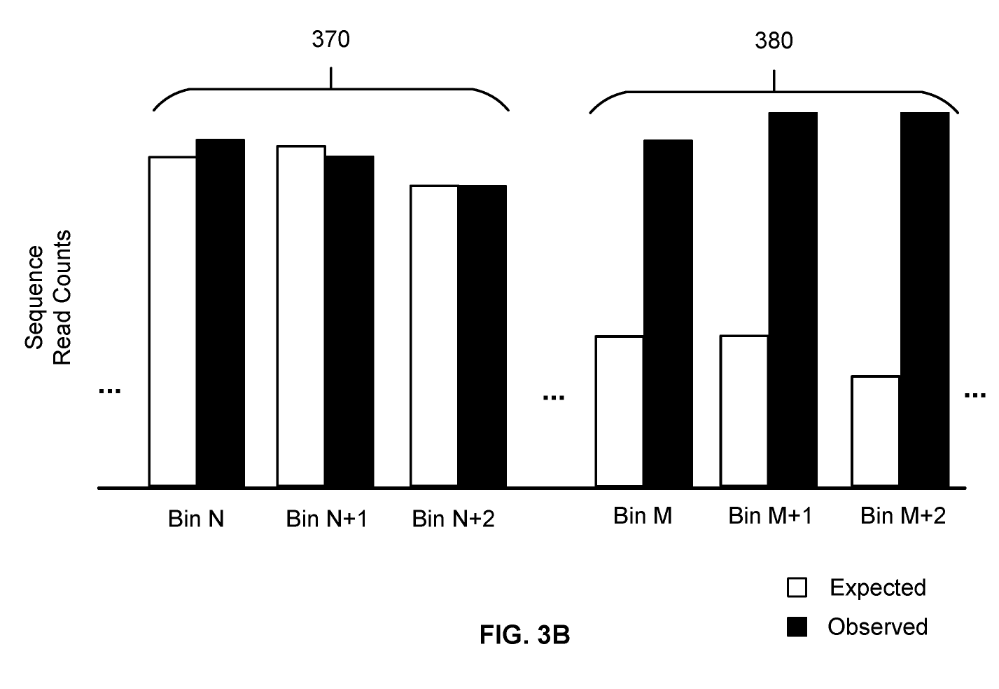

At step 135, a CNA is identified using the aligned sequence reads. A CNA is indicative of a somatic tumor event and can be informative for predicting a presence of cancer. In some embodiments, a CNA is identified using aligned sequence reads that are sequenced from nucleic acids extracted from a single sample, such as a cfDNA sample. In some embodiments, a CNA is identified using aligned sequence reads that are sequenced from nucleic acids extracted from multiple samples, such as a cfDNA sample and a gDNA sample. For example, aligned sequence reads derived from a gDNA sample can be used to identify germline or somatic non-tumor events such that corresponding events determined from aligned sequence reads derived from a cfDNA sample are not mistakenly interpreted as CNAs. The process for identifying CNAs is described in further detail below in reference to FIGS. 2A, 2B, 3A, and 3B.

Identifying Copy Number Aberrations

FIG. 2A is an example flow process 135 for identifying a source of a copy number event identified in a cfDNA sample, in accordance with an embodiment. Specifically, FIG. 2A depicts additional steps of step 135 shown in FIG. 1 for detecting a CNA in an individual.

At step 205, aligned sequence reads derived from a cfDNA sample (hereafter referred to as cfDNA sequence reads) and aligned sequence reads derived from a gDNA sample (hereafter referred to as gDNA sequence reads) are obtained.

At step 210, the aligned cfDNA sequence reads and gDNA sequence reads are analyzed to identify statistically significant bins and segments across a reference genome for each of the cfDNA sample and gDNA sample, respectively. A bin includes a range of nucleotide bases of a genome. A segment refers to one or more bins. Therefore, each sequence read is categorized in bins and/or segments that include a range of nucleotide bases that corresponds to the sequence read. Each statistically significant bin or segment of the genome includes a total number of sequence reads categorized in the bin or segment that is indicative of a copy number event. Generally, a statistically significant bin or segment includes a sequence read count that significantly differs from an expected sequence read count for the bin or segment even when accounting for possibly confounding factors, examples of which includes processing biases, variance in the bin or segment, or an overall level of noise in the sample (e.g., cfDNA sample or gDNA sample). Therefore, the sequence read count of a statistically significant bin and/or a statistically significant segment likely indicates a biological anomaly such as a presence of a copy number event in the sample.

Step 210 includes both a bin-level analysis to identify statistically significant bins as well as a segment-level analysis to identify statistically significant segments. Performing analyses at the bin and segment level enables the more accurate identification of possible copy number events. In some embodiments, solely performing an analysis at the bin level may not be sufficient to capture copy number events that span multiple bins. In other embodiments, solely performing an analysis at the segment level may yield an analysis that is not sufficiently granular enough to capture copy number events whose size are on the order of individual bins.

Generally, the analysis of cfDNA sequence reads and the analysis of gDNA sequence reads are conducted independent of one another. In various embodiments, the analysis of cfDNA sequence reads and gDNA sequence reads are conducted in parallel. In some embodiments, the analysis of cfDNA sequence reads and gDNA sequence reads are conducted at separate times depending on when the sequence reads are obtained (e.g., when sequence reads are obtained in step 205). Reference is now made to FIG. 2B, which is an example flow process that describes the analysis for identifying statistically significant bins and statistically significantly segments derived from cfDNA and gDNA samples, in accordance with an embodiment. Specifically, FIG. 2B depicts steps included in step 210 shown in FIG. 2. Therefore, steps 220-260 can be performed for a cfDNA sample and similarly, steps 220-260 can be separately performed for a gDNA sample.

At step 220, a bin sequence read count is determined for each bin of a reference genome. Generally, each bin represents a number of contiguous nucleotide bases of the genome. A genome can be composed of numerous bins (e.g., hundreds or even thousands). In some embodiments, the number of nucleotide bases in each bin is constant across all bins in the genome. In some embodiments, the number of nucleotide bases in each bin differs for each bin in the genome. In one embodiment, the number of nucleotide bases in each bin is between 25 kilobases (kb) and 10,000 kilobases (kb). In one embodiment, the number of nucleotide bases in each bin is between 50 kilobases kb) and 1000 kilobases (kb). In one embodiment, the number of nucleotide bases in each bin is between 100 kilobases (kb) and 500 kb. In one embodiment, the number of nucleotide bases in each bin is between 50 kb and 100 kb. In one embodiment, the number of nucleotide bases in each bin is between 45 kb and 75 kb. In one embodiment, the number of nucleotide bases in each bin is 50 kb. In practice, other bin sizes may be used as well.

The bin sequence read count of a bin represents a total number of sequence reads that are categorized in the bin. A sequence read is categorized in a bin if the sequence read spans a threshold number of nucleotide bases that are included in the bin (i.e., align or map to a bin). In one embodiment, each sequence read categorized in a bin spans at least one nucleotide base that is included in the bin. Reference is now made to FIG. 3A, which is an example depiction of sequence reads 330 in relation to bins 320 of a reference genome 305, in accordance with an embodiment. Sequence read 330A, sequence read 330B, and sequence read 330C can each include a different number of nucleotide bases and can span one or more of the bins 320.

As shown in FIG. 3A, sequence read 330A includes fewer nucleotide bases in comparison to the number of nucleotide bases in a bin (e.g., bin 320B). Here, sequence read 330A is categorized in bin 320B. Sequence read 330B spans nucleotide bases that are included in both bin 320C and bin 320D. Therefore, sequence read 330B is categorized in both bin 320C and bin 320D. Sequence read 330C spans nucleotide bases that are included in bin 320B, bin 320C, and bin 320D. Therefore, sequence read 330C is categorized in each of bin 320B, bin 320C, and bin 320D.

To determine the bin sequence read count for each bin, the sequence reads categorized in each bin are quantified. Therefore, bin 320A shown in FIG. 3A has a bin sequence read count of zero, bin 320B has a bin sequence read count of two (e.g., sequence read 330A and sequence read 330C), bin 320C has a bin sequence read count of two (e.g., sequence read 330B and sequence read 330C), bin 320D has a bin sequence read count of two (e.g., sequence read 330B and sequence read 330C), and bin 320E has a bin sequence read count of one (e.g., sequence read 330C).

Returning to FIG. 2B, at step 225, the bin sequence read count for each bin is normalized to remove one or more different processing biases. Generally, the bin sequence read count for a bin is normalized based on processing biases that were previously determined for the same bin. In one embodiment, normalizing the bin sequence read count involves dividing the bin sequence read count by a value representing the processing bias. In one embodiment, normalizing the bin sequence read count involves subtracting a value representing the processing bias from the bin sequence read count. Examples of a processing bias for a bin can include guanine-cytosine (GC) content bias, mappability bias, or other forms of bias captured through a principal component analysis. Processing biases for a bin can be accessed from the processing biases store 270 shown in FIG. 2C.

At step 230, a bin score for each bin is determined by modifying the bin sequence read count for the bin by the expected bin sequence read count for the bin. Step 230 serves to normalize the observed bin sequence read count such that if the particular bin consistently has a high sequence read count (e.g., high expected bin sequence read counts) across many samples, then the normalization of the observed bin sequence read count accounts for that trend. The expected sequence read count for the bin can be accessed from the bin expected counts store 280 in the training characteristics database 265 (see FIG. 2C). The generation of the expected sequence read count for each bin is described in further detail below.

In one embodiment, a bin score for a bin can be represented as the log of the ratio of the observed sequence read count for the bin and the expected sequence read count for the bin. For example, bin score b₁ for bin i can be expressed as:

$${b}_{i} = log ( \frac{observed bin sequence read count}{expected bin sequence read count} ) \qquad ( 1 )$$

In other embodiments, the bin score for the bin can be represented as the ratio between the observed sequence read count for the bin and the expected sequence read count for the bin (e.g.,

$$\frac{observed}{expected} ) ,$$

the square root of the ratio (e.g.,

$$\sqrt{\frac{observed}{expected}} ) ,$$

a generalized log transformation (glog) of the ratio (e.g., log(observed+{square root over (observed²+expected))}) or other variance stabilizing transforms of the ratio.

Reference is now made to FIG. 3B, which is an example chart depicting expected and observed sequence read counts across different bins of a reference genome, in accordance with an embodiment. Specifically, FIG. 3B depicts observed and expected sequence read counts for a first set 370 of bins (e.g., Bin N, Bin N+1, Bin N+2) and for a second set 380 of bins (e.g., Bin M, Bin M+1, Bin M+2). In various embodiments, bins in the first set 370 may be from a first segment of the reference genome whereas bins in the second set 380 may be from a second segment of the reference genome. In some embodiments, bins in the first set 370 may be from a first chromosome whereas bins in second set 380 are from a different chromosome.

Here, the observed sequence read counts and expected sequence read counts for bins in the first set 370 may not differ significantly. However, the observed sequence read counts for bins in the second set 380 may be significantly higher than the corresponding expected read counts for the bins. Therefore, the bin scores for each of the bins in the second set 380 are higher than the bin scores for each of the bins in the first set 370. The higher bin scores of the bins in the second set 380 indicate a higher likelihood that the observed sequence read counts in bin M, bin M+1, and bin M+2 are a result of a copy number event.

The differing bin scores for the first set 370 and second set 380 of bins illustrates the benefit of normalizing the observed sequence read counts for each bin by the corresponding expected sequence read counts for the bin. Specifically, in the example shown in FIG. 3B, the observed sequence read counts for bins in the first set 370 and the observed sequence read counts for bins in the second set 380 may not significantly differ from each other. By modifying the observed sequence read counts to account for expected sequence read counts, a possible copy number event that corresponds to the second set 380 of bins can be identified.

Returning to FIG. 2B, at step 235, a bin variance estimate is determined for each bin. Here, the bin variance estimate represents an expected variance for the bin that is further adjusted by an inflation factor that represents a level of variance in the sample. Put another way, the bin variance estimate represents a combination of the expected variance of the bin that is determined from prior training samples as well as an inflation factor of the current sample (e.g., cfDNA or gDNA sample) which is not accounted for in the expected variance of the bin.

To provide an example, a bin variance estimate (varᵢ) for a bin i can be expressed as:

varᵢ=varₑₓₚ_{ᵢ}*Iₛₐₘₚₗₑ (2)

where varₑₓₚ_{ᵢ} represents the expected variance of bin i determined from prior training samples and Iₛₐₘₚₗₑ represents the inflation factor of the current sample. Generally, the expected variance of a bin (e.g., varₑₓₚ) is obtained by accessing the bin expected variance store 290 shown in FIG. 2C.

To determine the inflation factor Iₛₐₘₚₗₑ of the sample, a deviation of the sample is determined and combined with sample variation factors that are retrieved from the sample variation factors store 295 shown in FIG. 2C. Sample variation factors are coefficient values that are previously derived by performing a fit across data derived from multiple training samples. For example, if a linear fit is performed, sample variation factors can include a slope coefficient and an intercept coefficient. If higher order fits are performed, sample variation factors can include additional coefficient values.

The deviation of the sample represents a measure of variability of sequence read counts in bins across the sample. In one embodiment, the deviation of the sample is a median absolute pairwise deviation (MAPD) and can be calculated by analyzing sequence read counts of adjacent bins. Specifically, the MAPD represents the median of absolute value differences between bin scores of adjacent bins across the sample. Mathematically, the MAPD can be expressed as:

∀(binᵢ, binᵢ₊₁), MAPD=median{|(bᵢ)−(bᵢ₊₁)|} (3)

where bᵢ and bᵢ₊₁ are the bin scores for bin i and bin i+1 respectively.

The inflation factor Iₛₐₘₚₗₑ is determined by combining the sample variation factors and the deviation of the sample (e.g., MAPD). As an example, the inflation factor Iₛₐₘₚₗₑ of a sample can be expressed as:

Iₛₐₘₚₗₑ=slope*σₛₐₘₚₗₑ+intercept. (4)

Here, each of the “slope” and “intercept” coefficients are sample variation factors accessed from the sample variation factors store 295 whereas σₛₐₘₚₗₑ represents the deviation of the sample.

At step 240, each bin is analyzed to determine whether the bin is statistically significant based on the bin score and bin variance estimate for the bin. For each bin i, the bin score (bᵢ) and the bin variance estimate (varᵢ) of the bin can be combined to generate a z-score for the bin. An example of the z-score (zᵢ) of bin i can be expressed as:

$${z}_{i} = \frac{{b}_{i}}{{var}_{i}} \qquad ( 5 )$$

To determine whether a bin is a statistically significant bin, the z-score of the bin is compared to a threshold value. If the z-score of the bin is greater than the threshold value, the bin is deemed a statistically significant bin. Conversely, if the z-score of the bin is less than the threshold value, the bin is not deemed a statistically significant bin. In one embodiment, a bin is determined to be statistically significant if the z-score of the bin is greater than 2. In other embodiments, a bin is determined to be statistically significant if the z-score of the bin is greater than 2.5, 3, 3.5, or 4. In one embodiment, a bin is determined to be statistically significant if the z-score of the bin is less than −2. In other embodiments, a bin is determined to be statistically significant if the z-score of the bin is less than −2.5, −3, −3.5, or −4. The statistically significant bins can be indicative of one or more copy number events that are present in a sample (e.g., cfDNA or gDNA sample).

At step 245, segments of the reference genome are generated. Each segment is composed of one or more bins of the reference genome and has a statistical sequence read count. Examples of a statistical sequence read count can be an average bin sequence read count, a median bin sequence read count, and the like. Generally, each generated segment of the reference genome possesses a statistical sequence read count that differs from a statistical sequence read count of an adjacent segment. Therefore, a first segment may have an average bin sequence read count that significantly differs from an average bin sequence read count of a second, adjacent segment.

In various embodiments, the generation of segments of the reference genome can include two separate phases. A first phase can include an initial segmentation of the reference genome into initial segments based on the difference in bin sequence read counts of the bins in each segment. The second phase can include a re-segmentation process that involves recombining one or more of the initial segments into larger segments. Here, the second phase considers the lengths of the segments created through the initial segmentation process to combine false-positive segments that were a result of over-segmentation that occurred during the initial segmentation process.

Referring more specifically to the initial segmentation process, one example of the initial segmentation process includes performing a circular binary segmentation algorithm to recursively break up portions of the reference genome into segments based on the bin sequence read counts of bins within the segments. In other embodiments, other algorithms can be used to perform an initial segmentation of the reference genome. As an example of the circular binary segmentation process, the algorithm identifies a break point within the reference genome such that a first segment formed by the break point includes a statistical bin sequence read count of bins in the first segment that significantly differs from the statistical bin sequence read count of bins in the second segment formed by the break point. Therefore, the circular binary segmentation process yields numerous segments, where the statistical bin sequence read count of bins within a first segment is significantly different from the statistical bin sequence read count of bins within a second, adjacent segment.

The initial segmentation process can further consider the bin variance estimate for each bin when generating initial segments. For example, when calculating a statistical bin sequence read count of bins in a segment, each bin i can be assigned a weight that is dependent on the bin variance estimate (e.g., varᵢ) for the bin. In one embodiment, the weight assigned to a bin is inversely related to the magnitude of the bin variance estimate for the bin. A bin that has a higher bin variance estimate is assigned a lower weight, thereby lessening the impact of the bin's sequence read count on the statistical bin sequence read count of bins in the segment. Conversely, a bin that has a lower bin variance estimate is assigned a higher weight, which increases the impact of the bin's sequence read count on the statistical bin sequence read count of bins in the segment.

Referring now to the re-segmenting process, it analyzes the segments created by the initial segmentation process and identifies pairs of falsely separated segments that are to be recombined. The re-segmentation process may account for a characteristic of segments not considered in the initial segmentation process. As an example, a characteristic of a segment may be the length of the segment. Therefore, a pair of falsely separated segments can refer to adjacent segments that, when considered in view of the lengths of the pair of segments, do not have significantly differing statistical bin sequence read counts. Longer segments are generally correlated with a higher variation of the statistical bin sequence read count. As such, adjacent segments that were initially determined to each have statistical bin sequence read counts that differed from the other can be deemed as a pair of falsely separated segments by considering the length of each segment.

Falsely separated segments in the pair are combined. Thus, performing the initial segmentation and re-segementing processes results in generated segments of a reference genome that takes into consideration variance that arises from differing lengths of each segment.

At step 250, a segment score is determined for each segment based on an observed segment sequence read count for the segment and an expected segment sequence read count for the segment. An observed segment sequence read count for the segment represents the total number of observed sequence reads that are categorized in the segment. Therefore, an observed segment read count for the segment can be determined by summating the observed bin read counts of bins that are included in the segment. Similarly, the expected segment sequence read count represents the expected sequence read counts across the bins included in the segment. Therefore, the expected segment sequence read count for a segment can be calculated by quantifying the expected bin sequence read counts of bins included in the segment. The expected read counts of bins included in the segment can be accessed from the bin expected counts store 280.

The segment score for a segment can be expressed as the ratio of the segment sequence read count and the expected segment sequence read count for the segment. In one embodiment, the segment score for a segment can be represented as the log of the ratio of the observed sequence read count for the segment and the expected sequence read count for the segment.

Segment score sₖ for segment k can be expressed as:

$${s}_{k} = log ( \frac{observed segment sequence read count}{expected segment sequence read count} ) \qquad ( 6 )$$

In other embodiments, the segment score for the segment can be represented as one of the square root of the ratio (e.g.,

$$\sqrt{\frac{observed}{expected}} ) ,$$

a generalized log transformation of the ratio (e.g., log (observed+{square root over (observed²+expected))}) or other variance stabilizing transforms of the ratio.

At step 255, a segment variance estimate is determined for each segment. Generally, the segment variance estimate represents how deviant the sequence read count of the segment is. In one embodiment, the segment variance estimate can be determined by using the bin variance estimates of bins included in the segment and further adjusting the bin variance estimates by a segment inflation factor (I_{segment}). To provide an example, the segment variance estimate for a segment k can be expressed as:

varₖ=mean(varᵢ)*I_{segment} (7)

where mean(varᵢ) represents the mean of the bin variance estimates of bins i that are included in segment k. The bin variance estimates of bins can be obtained by accessing the bin expected variance store 290.

The segment inflation factor accounts for the increased deviation at the segment level that is typically higher in comparison to the deviation at the bin level. In various embodiments, the segment inflation factor may scale according to the size of the segment. For example, a larger segment composed of a large number of bins would be assigned a segment inflation factor that is larger than a segment inflation factor assigned to a smaller segment composed of fewer bins. Thus, the segment inflation factor accounts for higher levels of deviation that arises in longer segments. In various embodiments, the segment inflation factor assigned to a segment for a first sample differs from the segment inflation factor assigned to the same segment for a second sample. In various embodiments, the segment inflation factor I_{segment} for a segment with a particular length can be empirically determined in advance.

In various embodiments, the segment variance estimate for each segment can be determined by analyzing training samples. For example, once the segments are generated in step 245, sequence reads from training samples are analyzed to determine an expected segment sequence read count for each generated segment and an expected segment variance estimate for each segment.

The segment variance estimate for each segment can be represented as the expected segment variance estimate for each segment determined using the training samples adjusted by the sample inflation factor. For example, the segment variance estimate (varₖ) for a segment k can be expressed as:

varₖ=varₑₓₚ_{ₖ}*Iₛₐₘₚₗₑ (8)

where varₑₓₚ_{ₖ} is the expected segment variance estimate for segment k and Iₛₐₘₚₗₑ is the sample inflation factor described above in relation to step 235 and Equation (4).

At step 260, each segment is analyzed to determine whether the segment is statistically significant based on the segment score and segment variance estimate for the segment. For each segment k, the segment score (sₖ) and the segment variance estimate (varₖ) of the segment can be combined to generate a z-score for the segment. An example of the z-score (zₖ) of segment k can be expressed as:

$${z}_{k} = \frac{{s}_{k}}{{var}_{k}} \qquad ( 9 )$$

To determine whether a segment is a statistically significant segment, the z-score of the segment is compared to a threshold value. If the z-score of the segment is greater than the threshold value, the segment is deemed a statistically significant segment. Conversely, if the z-score of the segment is less than the threshold value, the segment is not deemed a statistically significant segment. In one embodiment, a segment is determined to be statistically significant if the z-score of the segment is greater than 2. In other embodiments, a segment is determined to be statistically significant if the z-score of the segment is greater than 2.5, 3, 3.5, or 4. In some embodiments, a segment is determined to be statistically significant if the z-score of the segment is less than −2. In other embodiments, a segment is determined to be statistically significant if the z-score of the segment is less than −2.5, −3, −3.5, or −4. The statistically significant segments can be indicative of one or more copy number events that are present in a sample (e.g., cfDNA or gDNA sample).

Returning to FIG. 2A, at step 215, a source of a copy number event indicated by statistically significant bins (e.g., determined at step 240) and/or statistically significant segments (e.g., determined at step 260) derived from the cfDNA sample is determined. Specifically, statistically significant bins of the cfDNA sample are compared to corresponding bins of the gDNA sample. Additionally, statistically significant segments of the cfDNA sample are compared to corresponding segments of the gDNA sample.

The comparison between statistically significant segments and bins of the cfDNA sample and corresponding segments and bins of the gDNA sample yields a determination as to whether the statistically significant segments and bins of the cfDNA sample align with the corresponding segments and bins of the gDNA sample. As used hereafter, aligned segments or bins refers to the fact that the segments or bins are statistically significant in both the cfDNA sample and the gDNA sample. On the contrary, unaligned or not aligned segments or bins refers to the fact that the segments or bins are statistically significant in one sample (e.g., cfDNA sample), but is not statistically significant in another sample (e.g., gDNA sample).

Generally, if statistically significant bins and statistically significant segments of the cfDNA sample are aligned with corresponding bins and segments of the gDNA sample that are also statistically significant, this indicates that the same copy number event is present in both the cfDNA sample and the gDNA sample. Therefore, the source of the copy number event is likely to be due to a non-tumor event (e.g., either a germline or somatic non-tumor event) and the copy number event is likely a copy number variation.

Conversely, if statistically significant bins and statistically significant segments of the cfDNA sample are aligned with corresponding bins and segments of the gDNA sample that are not statistically significant, this indicates that the copy number event is present in the cfDNA sample but is absent from the gDNA sample. In this scenario, the source of the copy number event in the cfDNA sample is due to a somatic tumor event and the copy number event is a copy number aberration.

Identifying the source of a copy number event that is detected in the cfDNA sample is beneficial in filtering out copy number events that are due to a germline or somatic non-tumor event. This improves the ability to correctly identify copy number aberrations that are due to the presence of a solid tumor.

Determining Training Characteristics

FIG. 2C depicts an example database 265 that stores characteristics that are used to identify a source of a copy number event, in accordance with an embodiment. Specifically, the training characteristics database 265 can include a processing biases store 270, a bin expected counts store 280, a bin expected variance store 290, and a sample variation factors store 295. Each store 270, 280, 290, and 295 can include characteristics that are derived from training samples. In various embodiments, training samples are obtained from a healthy individual. In some embodiments, a training sample includes both a training cfDNA sample and a training gDNA sample. Each training cfDNA sample and training gDNA sample can be processed according to steps 105-130 shown in FIG. 1 to generate aligned cfDNA sequence reads and aligned gDNA sequence reads. As discussed hereafter, the aligned cfDNA sequence reads and aligned gDNA sequence reads derived from training samples can be used to determine characteristics that are stored in the training characteristics database 265.

The processing biases store 270 includes characteristics that represent a measure of a processing bias for each bin of the reference genome. In one embodiment, the processing biases store 270 can include, for each bin of the reference genome, 1) a GC content bias, 2) a mappability bias, and 3) information for determining a bias derived from a dimensionality reduction analysis. An example of a dimensionality reduction analysis is a principal component analysis (PCA). Additional processing biases for each bin can be included in the processing biases store 270. In various embodiments, the bins of the reference genome can be differently sized to minimize the effects of the processing biases that arise within each bin. For example, bins of the reference can be sized to more evenly distribute GC content amongst the bins, thereby minimizing differences in GC bias between different bins.

The GC content bias for a bin is based on a level of guanine-cytosine content within the bin. Generally, higher GC content within a bin leads to a higher number of bin sequence reads. Therefore, the processing biases store 270 can store a GC content bias for a bin that is directly correlated with the amount of GC content in the bin. During deployment, the GC content bias for the bin can be retrieved from the processing biases store 270 and a bin sequence read count for the bin can be normalized using the GC content bias for the bin. In various embodiments, the GC content bias for a bin can be determined using the GC content across smaller windows of the bin. For example, a window of a bin can be a range of nucleotide bases (e.g., 50, 100, 150 nucleotide bases). The GC content for the bin can be an average level of GC content across the windows of the bin.

The mappability bias for a bin is based on the mappability of the nucleotide base sequence of the bin. The mappability of nucleotide base sequences of a bin can be accessed from publicly available databases such as the UC Santa Cruz Genome Browser. Certain bins include nucleotide base sequences that have a higher mappability than other bins. Bins of higher mappability typically have higher bin sequence read counts. Therefore, the processing biases store 270 can store a mappability bias for a bin that is directly correlated with the mappability of the bin. During deployment, the mappability bias for the bin can be retrieved from the processing biases store 270 and a bin sequence read count for the bin can be normalized using the mappability bias for the bin. In various embodiments, the mappability for a bin can be determined using the mappability across smaller windows of the bin, such as windows described above in relation to the GC content bias. The mappability for the bin can be an average mappability across the windows of the bin.

The bias derived from a dimensionality reduction analysis can be a PCA bias. The PCA bias represents bias in a bin that can arise from unknown sources. Given training sequence reads (e.g., cfDNA sequence reads and/or gDNA sequence reads derived from training samples), a principal component analysis is performed to identify principal components PCₙ for bin sequence read counts s(i) for the bin i. The PCA analysis can be expressed as:

s(i)=a+b₁*PC₁(i)+ . . . +bₙ*PCₙ(i) (10)

Here, each of the parameters (a, b₁ . . . bₙ) and the principal components PCₙ are determined using the bin sequence read counts for the bin derived from the training examples. Furthermore, the parameters and the principal components can be stored in the processing biases store 270. During deployment, the parameters and principal components for the bin can be accessed to determine a PCA bias for the bin. Therefore, the bin sequence reads counts for the bin can be normalized by a PCA bias for the bin.

The bin expected counts store 280 holds the expected sequence read count for each bin across the genome. The expected sequence read count for each bin is determined using training sequence reads (e.g., cfDNA sequence reads and/or gDNA sequence reads derived from a training sample). Specifically, training sequence reads of a training sample are categorized into bins of the reference genome and the total number of training sequence reads in the bin is determined for the training sample. The expected sequence read count for the bin is calculated as the average of the number of training sequence reads categorized in the bin across multiple training samples.

The bin expected variance store 290 holds the expected variance for each bin in the genome. Generally, the expected variance for a bin is a measure of the variability of the sequence read count of the bin across training samples. As an example, the expected variance for a bin can be a standard deviation of the total number of training sequence reads categorized in the bin across multiple training samples. As another example, the expected variance for a bin can be a robust measure of the variability, such as a mean absolute deviation, of the sequence read count.

The sample variation factors store 295 holds factors that can be used to determine an inflation factor of a sample (e.g., Iₛₐₘₚₗₑ). Examples of factors stored in the sample variation factors store 295 include coefficient values that are determined through a curve fitting process that is performed on data derived from training samples.

More specifically, for each training sample, sequence reads from the training sample can be used to determine z-scores for each bin of the reference genome. A z-score for bin i can be expressed as:

$${z}_{i} = \frac{{b}_{i}}{{var}_{i}} \qquad ( 11 )$$

where bᵢ is the bin score for bin i and varᵢ is the bin variance estimate for the bin.

A first curve fit is performed between the bin z-scores of each training sample and the theoretical distribution of z-scores. Here, an example theoretical distribution of z-scores is a normal distribution. In one embodiment, the first curve fit is a linear robust regression fit which yields a slope value. Therefore, performing the first curve fit between bin z-scores of a training sample and the theoretical distribution of z-scores yields a slope value. The first curve fit is performed multiple times for multiple training samples to calculate multiple slope values.

A second curve fit is performed between slope values and deviations of training samples. As an example, the deviation of a training sample can be a median absolute pairwise deviation (MAPD), which represents the median of absolute value differences between bin scores of adjacent bins across the training sample. In one embodiment, the second curve fit is a linear robust regression fit. In another embodiment, the second curve fit can be a higher order polynomial fit. The second curve fit yields coefficient values which, in the embodiment where the second curve fit is a linear robust regression fit, includes a slope coefficient and an intercept coefficient. The coefficient values yielded by the second curve fit are stored as sample variation factors in the sample variation factors store 295.

### EXAMPLES

### Example 1

### Copy Number Aberrations Originate from Somatic Tumor Source in a Cancer Sample

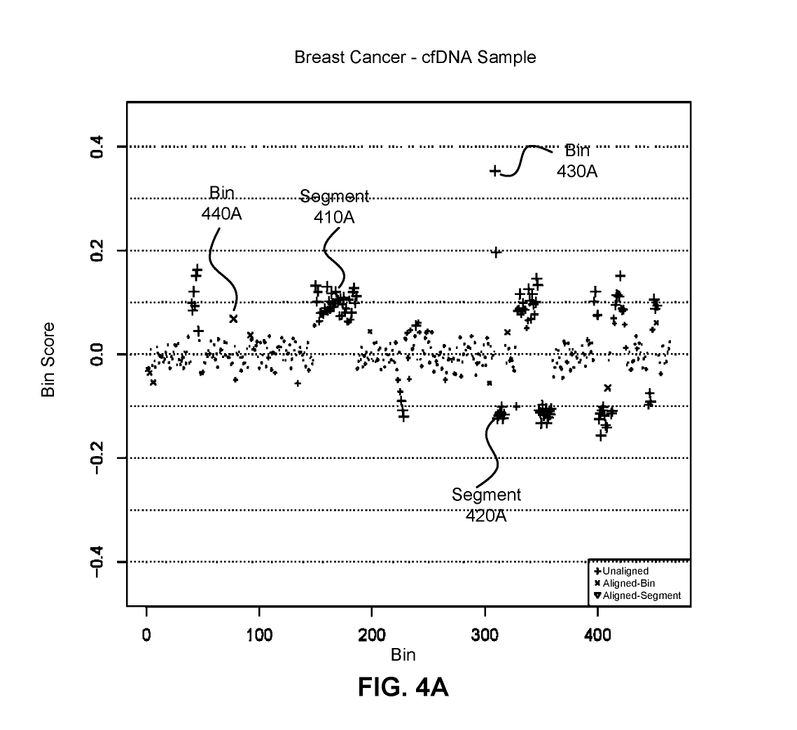

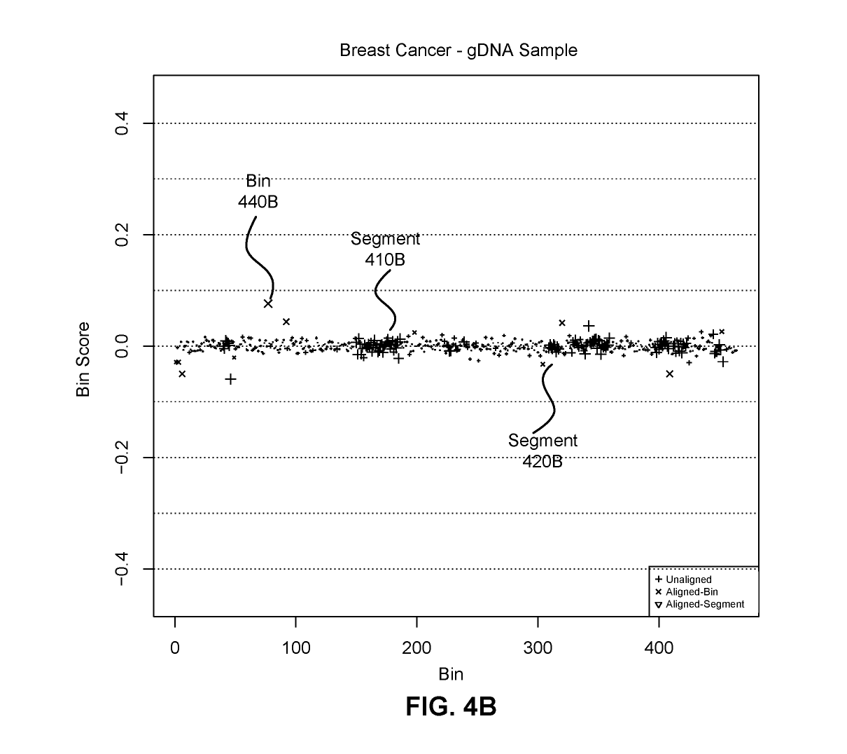

FIG. 4A and FIG. 4B depicts bin scores across a plurality of bins of a genome for a cfDNA sample and a gDNA sample, respectively, that are obtained from a cancer subject. Here, the cancer patient has been clinically diagnosed with stage 1 breast cancer. A blood test sample was obtained through a blood draw from the cancer patient and collected in a blood collection tube. The blood sample tube was centrifuged at 1600 g, the plasma and buffy coat components extracted, respectively, and stored at minus 20° C. cfDNA was extracted from plasma using QIAAMP Circulating Nucleic Acid kit (Qiagen, Germantown, Md.) and pooled. White blood cells in the buffy coat were lysed and gDNA extracted using a DNEASY Blood and Tissue kit (Qiagen, Germantown, Md.). Sequencing libraries were prepared from both the extracted cfDNA sample and the gDNA sample using TRUSEQ Nano DNA reagents (Illumina, San Diego, Calif.). After library preparation the cfDNA sequencing library and gDNA sequencing library were sequenced using a HiSeqX sequencer (Illumina, San Diego, Calif.) to obtain sequence reads from both the cfDNA and gDNA samples as described above in relation to step 125. Specifically, cfDNA sequence reads and gDNA sequence reads were obtained by performing whole genome sequencing at a depth of coverage of 35x. Sequence reads for each DNA sample were aligned and analyzed using the flow process 135 shown in FIG. 2A which further includes corresponding flow process 210 shown in FIG. 2B.

Referring specifically to the data shown in FIG. 4A and FIG. 4B, each indicator in each of the graphs of FIG. 4A and FIG. 4B represents a bin score for a bin of the reference genome. The select bins shown on the x-axis represent nucleotide sequences from chromosomes 1-22 of the cancer patient. The bin score for each bin is normalized relative to the number of sequence read counts expected for the bin and therefore, a cfDNA sample or a gDNA sample that is devoid of a copy number event would depict bin scores that minimally deviate from zero.

Unaligned indicators (e.g., marked as “+” in FIG. 4A and FIG. 4B) refer to bins and/or segments of the cfDNA sample that are different from corresponding bins and/or segments of the gDNA sample. For example, a statistically significant bin of the cfDNA sample is depicted as an unaligned indicator in FIG. 4A if the corresponding bin of the gDNA sample is not statistically significant. Similarly, a non-statistically significant bin of the cfDNA sample is depicted as an unaligned indicator in FIG. 4A if the corresponding bin of the gDNA sample is statistically significant. Additionally, all bins within a segment of a cfDNA sample are depicted using unaligned indicators if the segment of the cfDNA sample is different (e.g., statistically significant vs non-statistically significant) from the corresponding segment of the gDNA sample.

Aligned bin indicators (e.g., marked as “x” in FIG. 4A and FIG. 4B) refer to bins in the cfDNA sample and the gDNA sample that align. For example, a statistically significant bin of the cfDNA sample is depicted as an aligned bin indicator if the corresponding bin of the gDNA sample is also statistically significant. Similarly, a non-statistically significant bin of the cfDNA sample is depicted as an aligned bin indicator if the corresponding bin of the gDNA sample is also non-statistically significant.

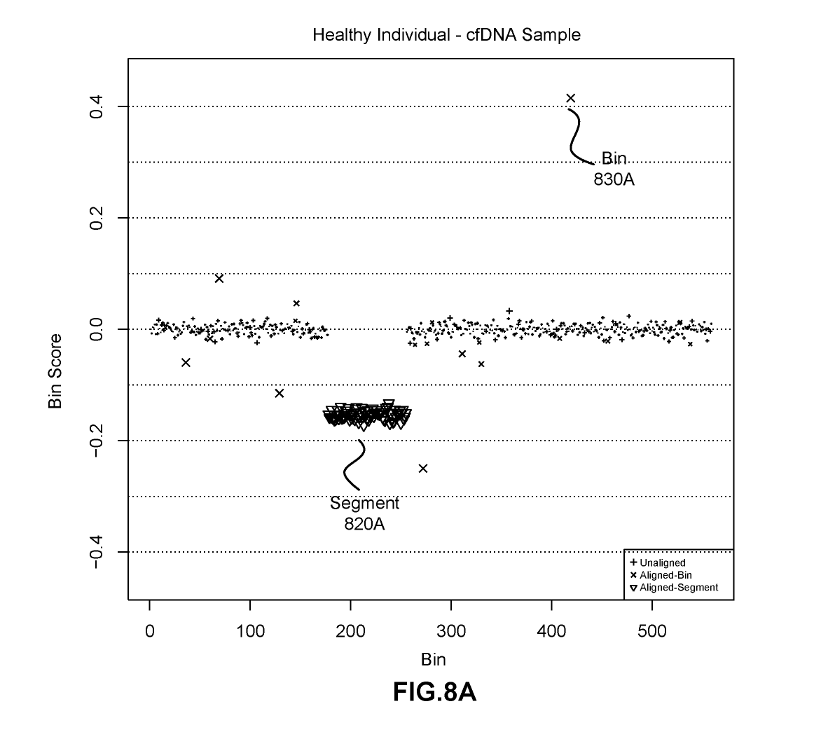

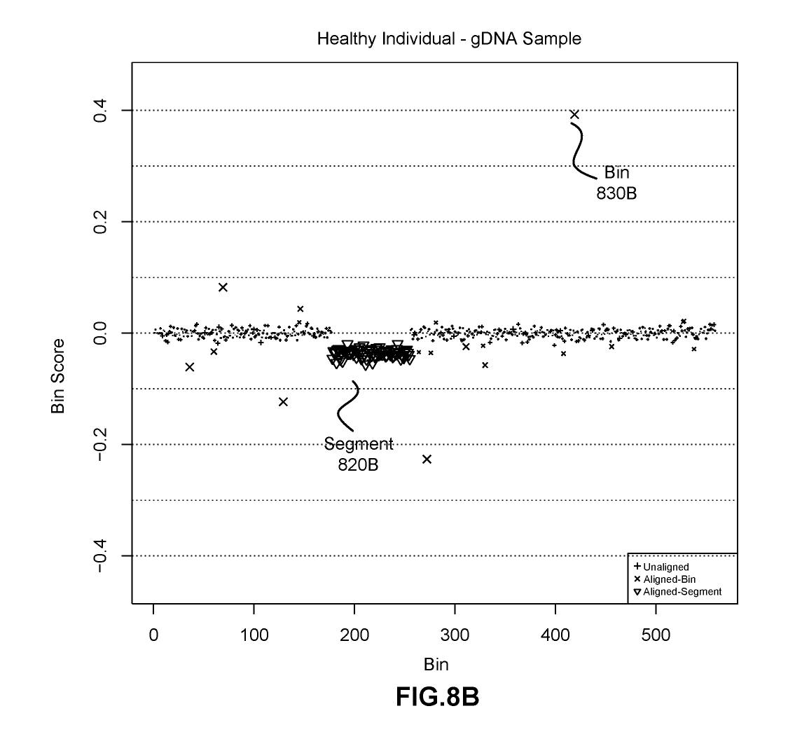

Aligned segment indicators (e.g., marked as “∇” in FIG. 4A and FIG. 4B) refer to bins in the cfDNA sample and the gDNA sample that are included in aligned segments. Specifically, the bins in a statistically significant segment of the cfDNA sample are depicted using aligned segment indicators if the corresponding segment of the gDNA sample is also statistically significant. Here, the bins in the corresponding segment of the gDNA sample are also depicted using aligned segment indicators. An example is shown in FIGS. 8A and 8B.

Referring to FIG. 4A, the cfDNA sample includes a statistically significant segment 410A that includes bins with bin scores above zero. Additionally, the cfDNA sample includes a statistically significant segment 420A that includes bins with bin scores below zero. Furthermore, the cfDNA sample includes bins 430A and 440A that are statistically significant as they each have a bin score that is above zero. Each statistically significant segment (e.g., 410A and 420A) and statistically significant bin (e.g., 430A and 440A) are indicative of a copy number event.

Referring to FIG. 4B, the gDNA sample includes segment 410B and segment 420B that each includes bins with bin scores that are not significantly different from a value of zero. Here, segment 410B of the gDNA sample is the corresponding segment of segment 410A of the cfDNA sample. Additionally, segment 420B of the gDNA sample is the corresponding segment of segment 420A of the cfDNA sample. The gDNA sample also includes statistically significant bin 440B that is the corresponding bin for bin 440A of the cfDNA sample.

Here, the statistically significant segments (e.g., segment 410A and 420A) in the cfDNA sample are unaligned with the corresponding segments (e.g., segment 410B and 420B) in the gDNA sample. Specifically, statistically significant segment 410A of the cfDNA sample is unaligned with segment 410B of the gDNA sample. Additionally, segment 420A of the cfDNA sample is unaligned with segment 420B of the gDNA sample. This indicates that the copy number events represented by each of the statistically significant segment 410A and 420B are likely due to a somatic tumor event.

Additionally, bin 430A of the cfDNA sample is unaligned with the corresponding bin of the gDNA sample (not shown) whereas bin 440A of the cfDNA sample aligns with bin 440B of the gDNA sample. Thus, the copy number event represented by bin 430A of the cfDNA sample is likely due to a somatic tumor event whereas the copy number event represented by bin 430B of the cfDNA sample is likely due to either a germline or somatic non-tumor event.

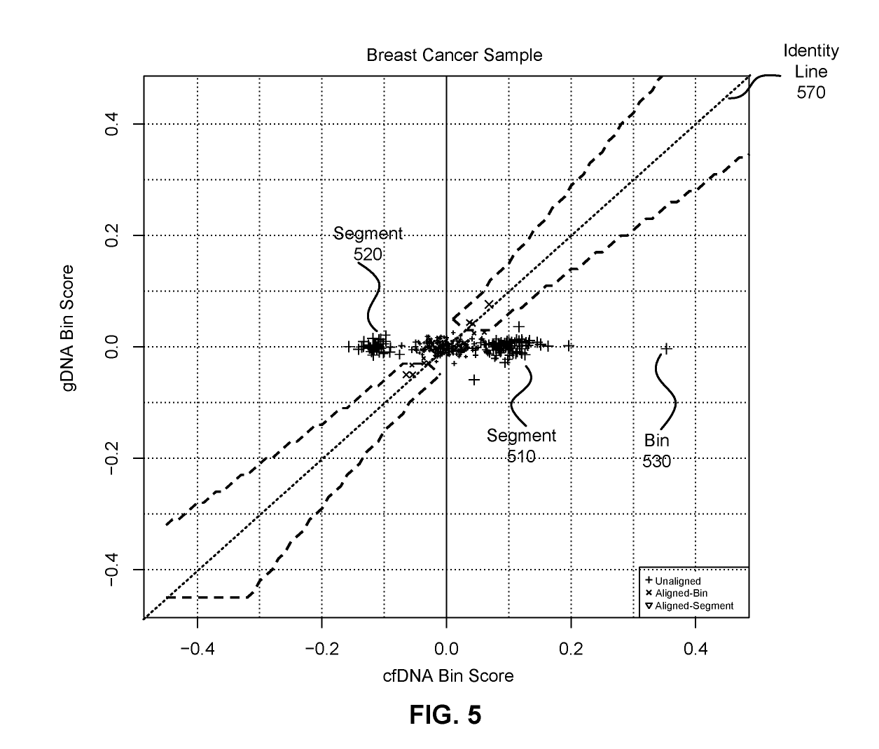

FIG. 5 is a graph depicting the distribution of bin scores for the gDNA sample shown in FIG. 4B in relation to corresponding bin scores for the cfDNA sample shown in FIG. 4A. In particular, FIG. 5 depicts a theoretical identity line 570 (e.g., y=x line) where the x-axis represents the bin scores for bins in the cfDNA sample and the y-axis represents bin scores of bins in the gDNA sample.

As shown in FIG. 5, statistically significant segment 510 (which represents segment 410A and 410B shown in FIG. 4A and FIG. 4B), statistically significant segment 520 (which represents segment 420A and 420B shown in FIG. 4A and FIG. 4B), and statistically significant bin 530 (which corresponds to bin 430A and 430B shown in FIG. 4A and FIG. 4B) deviate from the identity line 570. This is one method of visualizing the unalignment between statistically significant bins and segments of the cfDNA sample and corresponding bins and segments of the gDNA sample.

### Example 2

### Potential Copy Number Aberration Originates from Somatic Tumor Source in a Non-Cancer Sample

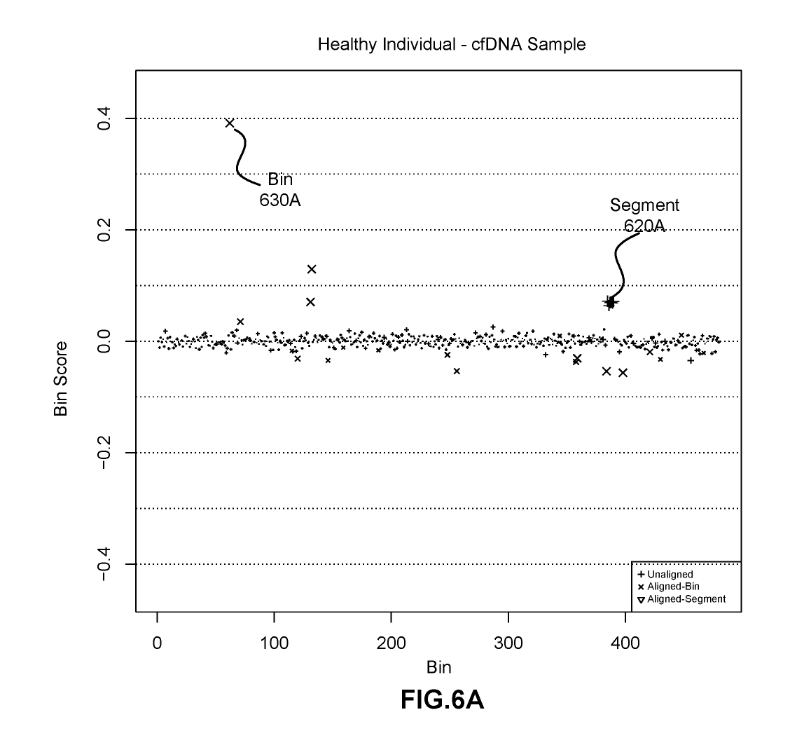

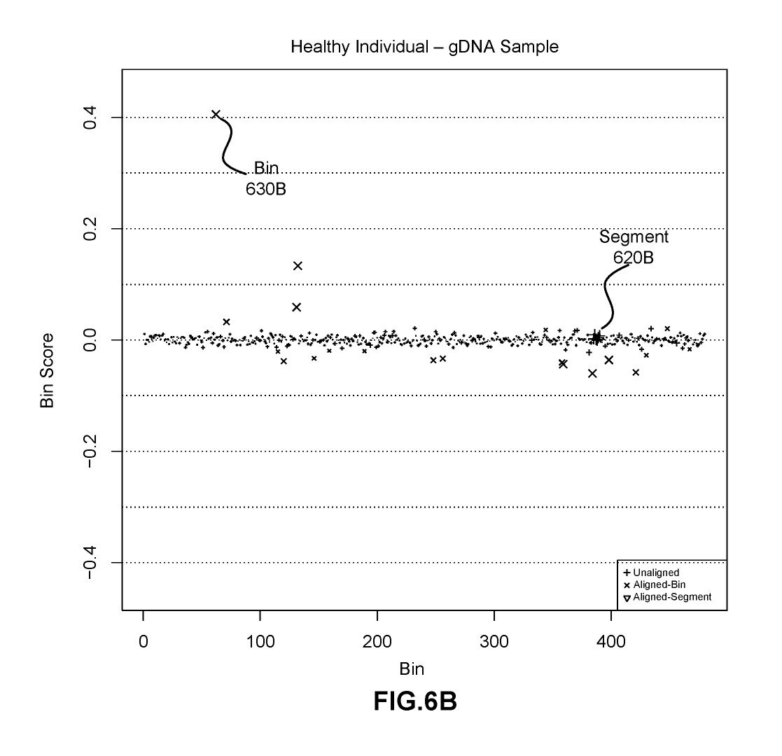

FIG. 6A and FIG. 6B depicts bin scores across bins of a genome determined from a cfDNA sample and a gDNA sample, respectively, that are obtained from a non-cancer individual. Here, as the individual has not been diagnosed with cancer, the individual can be a candidate for early detection of cancer. A blood test sample was obtained through a blood draw from the non-cancer individual and cfDNA and gDNA was extracted. Extraction and sequencing of cfDNA and gDNA samples to generate sequence reads for analysis was performed according to the process described above in Example 1.

As shown in FIG. 6A, the cfDNA sample includes a statistically significant segment 610A that includes bins with bin scores above zero. Additionally, the cfDNA sample includes a statistically significant bin 630A that includes a bin score above zero. The statistically significant segment 620A and statistically significant bin 630A are indicative of copy number events. As shown in FIG. 6B, the gDNA sample includes segment 620B that includes bins with bin scores that are not significantly different from a value of zero. Segment 620B of the gDNA sample is the corresponding segment of segment 620A of the cfDNA sample. Additionally, the gDNA sample also includes statistically significant bin 630B that is the corresponding bin for bin 630A of the cfDNA sample.

Bin 630A of the cfDNA sample aligns with bin 630B of the gDNA sample. Thus, the copy number event represented by bin 630A of the cfDNA sample is likely due to either a germline or somatic non-tumor event. The statistically significant segment 620A in the cfDNA sample is unaligned with the corresponding segment 620B in the gDNA sample. This indicates that the copy number event represented by the statistically significant segment 620A is possibly due to a somatic tumor event. This demonstrates that a healthy individual (e.g., not diagnosed for cancer) can potentially be screened for early detection of cancer by identifying possible copy number aberrations using cfDNA and gDNA samples obtained from the individual.

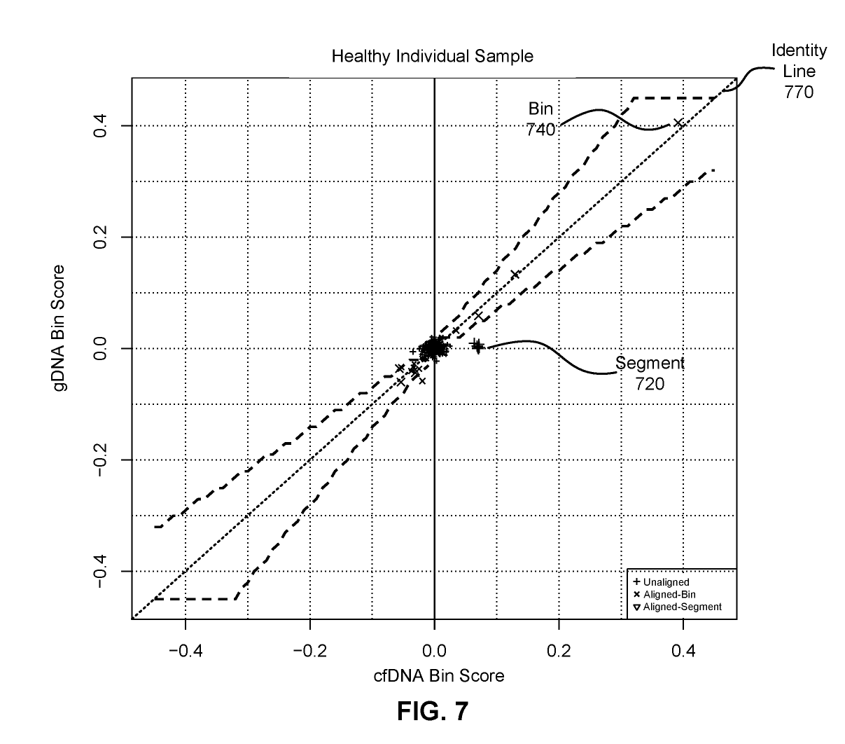

FIG. 7 is a graph depicting the distribution of bin scores for the gDNA sample shown in FIG. 6B in relation to corresponding bin scores for the cfDNA sample shown in FIG. 6A. In particular, FIG. 7 depicts a theoretical identity line 770 (e.g., y=x line) where the x-axis represents the bin scores for bins in the cfDNA sample and the y-axis represents bin scores of bins in the gDNA sample. As shown in FIG. 7 statistically significant segment 720 (which represents segment 620A and 620B shown in FIG. 6A and FIG. 6B) deviates from the identity line 770, thereby reflecting the unaligned statistically significant segment of the cfDNA sample and a corresponding non-statistically significant segment of the gDNA sample. Additionally, bin 740 (which represents bins 640A and 640B in FIG. 6A and FIG. 6B) is near the identity line 770. This reflects that the higher bin score of bin 640A in the cfDNA sample is aligned with a higher bin score of bin 640B in the gDNA sample.

### Example 3

### Copy Number Variations Originate from a Germline or Somatic Non-tumor Source in a Non-Cancer Sample

FIG. 8A and FIG. 8B depicts bin scores across bins of a genome determined from a cfDNA sample and a gDNA sample, respectively, that are obtained from a non-cancer individual. Here, as the individual has not been diagnosed with cancer, the individual can be a candidate for early detection of cancer. A blood test sample was obtained through a blood draw from the non-cancer individual and cfDNA and gDNA was extracted. Extraction and sequencing of cfDNA and gDNA samples to generate sequence reads for analysis was performed according to the process described above in Example 1.

As shown in FIG. 8A, the cfDNA sample includes a statistically significant segment 820A that includes bins with bin scores below zero. Additionally, the cfDNA sample includes a statistically significant bin 830A that includes a bin score above zero. The statistically significant segment 820A and statistically significant bin 830A are indicative of copy number events. As shown in FIG. 8B, the gDNA sample includes segment 820B. Segment 820B of the gDNA sample is the corresponding segment of segment 820A of the cfDNA sample. Here, the statistically significant segment 820B includes at least a subset of bins with bin scores that do not deviate significantly from zero. In other words, the segment-level analysis enables the identification of a statistically significant segment 820B that includes a subset of bins that, individually, would not have been identified as statistically significant bins. This demonstrates the benefit of performing a segment-level analysis, in addition to performing a bin-level analysis, in order to identify copy number events. The gDNA sample additionally includes statistically significant bin 830B that is the corresponding bin for bin 830A of the cfDNA sample.

Here, the statistically significant segment 820A in the cfDNA sample aligns with the corresponding statistically significant segment 820B in the gDNA sample. This indicates that the copy number event represented by the statistically significant segment 820A is likely due to either a germline or somatic non-tumor event. Additionally, bin 830A of the cfDNA sample aligns with bin 830B of the gDNA sample. Thus, the copy number event represented by bin 830A of the cfDNA sample is also likely due to either a germline or somatic non-tumor event.

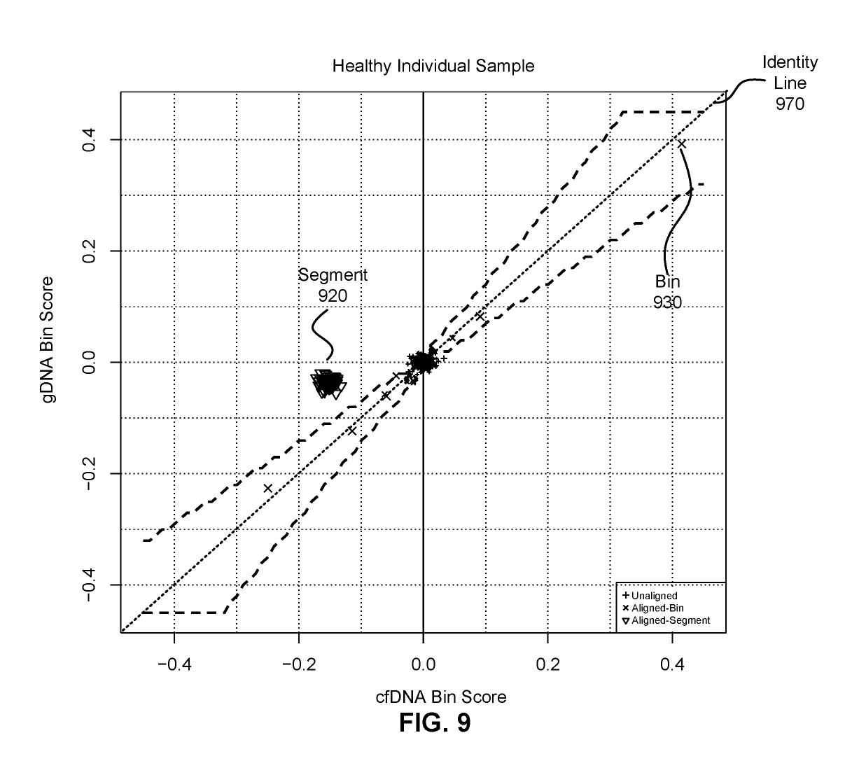

FIG. 9 is a graph depicting the distribution of bin scores for the gDNA sample shown in FIG. 8B in relation to corresponding bin scores for the cfDNA sample shown in FIG. 8A. In particular, FIG. 9 depicts a theoretical identity line 970 (e.g., y=x line) where the x-axis represents the bin scores for bins in the cfDNA sample and the y-axis represents bin scores of bins in the gDNA sample.

As shown in FIG. 9, bin 930 (which represents bins 830A and 830B in FIG. 8A and FIG. 8B) is near the identity line 970. This reflects that the higher bin score of bin 830A in the cfDNA sample is aligned with a similarly higher bin score of bin 830B in the gDNA sample.

Additionally, as shown in FIG. 9, statistically significant segment 920 (which represents the alignment between segments 820A and 820B shown in FIG. 8A and FIG. 8B) slightly deviates from the identity line 770. Here, although statistically significant segment 820A from the cfDNA sample aligns with statistically significant segment 820B from the gDNA sample, the slight deviation of segment 920 from the identity line 970 indicates that amount of deviation of the bin scores of bins in statistically significant segment 820A differs from the amount of deviation of the bins cores of bins in statistically significant segment 820B. For example, referring again to FIGS. 8A and 8B, the magnitude of bin scores of bins in segment 820A (e.g., magnitude ˜0.15 as shown in FIG. 8A) are greater than the magnitude of bin scores of bins in segment 820B (e.g., magnitude ˜0.05 as shown in FIG. 8B). This demonstrates that at the segment level, different samples can have different confounding factors that influence the bin scores in each segment. However, even in view of the different confounding factors in segment 820A and 820B, this example still demonstrates the ability to identify segments 820A and 820B as statistically significant segments.

### Additional Considerations

The foregoing detailed description of embodiments refers to the accompanying drawings, which illustrate specific embodiments of the present disclosure. Other embodiments having different structures and operations do not depart from the scope of the present disclosure. The term “the invention” or the like is used with reference to certain specific examples of the many alternative aspects or embodiments of the applicants' invention set forth in this specification, and neither its use nor its absence is intended to limit the scope of the applicants' invention or the scope of the claims. This specification is divided into sections for the convenience of the reader only. Headings should not be construed as limiting of the scope of the invention. The definitions are intended as a part of the description of the invention. It will be understood that various details of the present invention may be changed without departing from the scope of the present invention. Furthermore, the foregoing description is for the purpose of illustration only, and not for the purpose of limitation.

What is claimed is:

1. A method comprising:
obtaining sequence reads from a first sample and sequence reads from a second sample, each sequence read categorized in at least one bin of a plurality of bins of a genome;
for each of the first sample and the second sample;
for each bin in the plurality of bins of the genome:
determining a bin score by modifying a bin sequence read count to account for an expected sequence read count of the bin, the bin sequence read count representing a total number of sequence reads that are categorized in the bin;
determining a bin variance estimate for the bin;
determining whether the bin is statistically significant based on the bin score and the bin variance estimate for the bin;
generating segments of the genome that each include one or more bins in the plurality of bins,
for each generated segment of the genome:
determining a segment score for the segment based on a segment sequence read count for the segment, the segment sequence read count representing a total number of sequence reads that are categorized in bins included in the segment;
determining a segment variance estimate for the segment;
determining whether the segment is statistically significant based on the segment score and segment variance estimate for the segment; and
identifying a source of a copy number change in the first sample indicated by statistically significant bins and segments of the first sample by comparing each of at least one statistically significant bin and at least one statistically significant segment of the first sample to a corresponding at least one statistically significant bin and at least one statistically significant segment of the second sample.

2. The method of claim 1, wherein the first sample is a cfDNA sample and the second sample is a gDNA sample.

3. The method of claim 1, wherein determining a bin variance estimate for a bin comprises:
calculating a sample inflation factor representing a level of variance in the sample; and
adjusting an expected bin variance estimate for the bin by the sample inflation factor, the expected bin variance estimate for the bin determined from training samples.

4. The method of claim 3, wherein calculating the sample inflation factor comprises:
accessing one or more sample variation factors, the one or more sample variation factors previously derived by performing a fit operation across variations of training samples;
calculating a deviation score for the sample that represents a measure of variability of sequence read counts in bins across the sample; and
combining the one or more sample variation factors and the deviation of the sample to produce the sample inflation factor.

5. The method of claim 4, wherein the deviation of the sample is a median absolute pairwise deviation of sequence read counts of adjacent bins across the sample.

6. The method of claim 1, wherein determining whether the bin is statistically significant based on the bin score and the bin variance estimate for the bin comprises:
determining that a ratio of the bin score to the bin variance estimate is greater than a threshold value.

7. The method of claim 6, wherein the threshold value is 2.

8. The method of claim 1, wherein each generated segment of the genome has a statistical bin sequence read count across the one or more bins included in the segment that is different from a statistical bin sequence read count across bins included in an adjacent segment.

9. The method of claim 1, wherein generating segments of the genome that each include one or more bins in the plurality of bins comprises:
generating a plurality of initial segments of the genome; and
resegmenting the initial segments of the genome based on variances corresponding to lengths of each of the initial segments.

10. The method of claim 9, wherein resegmenting the initial segments of the genome comprises:
identifying a pair of falsely separated segments in the plurality of initial segments, the pair of falsely separated segments having bin sequence read counts within a threshold of each other; and
combining the pair of falsely separated segments.

11. The method of claim 9, wherein generating a plurality of initial segments of the genome comprises:
assigning a weight to each bin in the plurality of bins, the weight assigned to each bin being inversely related to the bin variance estimate for the bin; and
determining a statistical bin sequence read count of an initial segment based on at least the assigned weight to each bin in the initial segment.

12. The method of claim 1, wherein determining a segment score for a segment based on a segment sequence read count for the segment comprises:
determining an expected segment sequence read count by quantifying expected bin sequence read counts; and
determining a ratio between the segment sequence read count and the expected segment sequence read count.

13. The method of claim 1, wherein determining a segment variance estimate for a segment comprises:
determining a mean bin variance estimate across bins included in the segment; and
adjusting the mean bin variance estimate by a segment inflation factor.

14. The method of claim 1, wherein determining a segment variance estimate for a segment comprises:
determining an expected segment variance estimate for the segment based on sequence read counts for the segment derived from training samples; and
adjusting the expected segment variance estimate by a sample inflation factor representing a level of variance in the sample.

15. The method of claim 1, wherein determining whether a segment is statistically significant based on a segment score and segment variance estimate for the segment comprises:
determining that a ratio of the segment score to the segment variance estimate is greater than a threshold value.

16. The method of claim 15, wherein the threshold value is 2.

17. The method of claims 1, wherein prior to modifying a bin sequence read count to account for an expected sequence read count of a bin, normalizing the bin sequence read count for the bin to remove processing biases associated with the bin.

18. The method of claim 17, wherein removing processing biases associated with the bin comprises removing one or more of GC bias, mappability bias, or a bias determined through a dimensionality reduction analysis.

19. The method of claim 1, wherein an identified source of a copy number change is one of a germline event, a somatic non-tumor event, or a somatic tumor event.

20. The method of claim 1, wherein identifying the source of the copy number change further comprises:
responsive to the comparison yielding an alignment between the one or more statistically significant bins or segments of the first sample and the corresponding one or more bins or segments of the second sample, determining that the source of the copy number change is one of a germline event or a somatic non-tumor event.

21. The method of claim 1, wherein identifying the source of the copy number change further comprises:
responsive to the comparison yielding a lack of alignment between the one or more statistically significant bins or segments of the first sample and the corresponding one or more bins or segments of the second sample, determining that the source of the copy number change is a somatic tumor event.

22. The method of claim 1, wherein a bin in the plurality of bins of the genome includes between 500 kilobases to 1000 kilobases.

23. The method of claim 1, wherein a bin in the plurality of bins of the genome includes between 100 kilobases to 500 kilobases.

24. The method of claim 1, wherein a bin in the plurality of bins of the genome includes between 50 kilobases to 100 kilobases.

25. The method of claim 1, wherein a bin in the plurality of bins of the genome includes less than 50 kilobases.

26. The method of claim 1, wherein obtaining sequence reads from the first sample and sequence reads from the second sample comprises performing whole genome sequencing on nucleic acids obtained from the first sample and nucleic acids obtained from the second sample.

27. The method of claim 1, wherein obtaining sequence reads from the first sample and sequence reads from the second sample comprises performing whole exome sequencing on nucleic acids obtained from the first sample and nucleic acids obtained from the second sample.

28. A method comprising:
obtaining sequence reads from a first sample and sequence reads from a second sample, each sequence read categorized in at least one bin of a plurality of bins of the genome;
for each of the first sample and the second sample:
for each bin in the plurality of bins of the genome, determining whether the bin is a statistically significant bin;
generating segments of the genome that each include one or more bins in the plurality of bins,
for each generated segment of the genome, determining whether the segment is a statistically significant segment; and
identifying a source of a copy number change in the first sample by comparing at least one statistically significant bin or statistically significant segment of the first sample to a corresponding at least one statistically significant bin or statistically significant segment of the second sample.

29. The method of claim 28, wherein determining whether a bin is a statistically significant bin comprises:
determining a bin score by modifying a bin sequence read count to account for an expected sequence read count of the bin, the bin sequence read count representing a total number of sequence reads that are categorized in the bin; and
determining a bin variance estimate for the bin,
wherein determining whether the bin is a statistically significant bin is based on the bin score and the bin variance estimate for the bin.

30. The method of claim 28, wherein determining whether a segment is a statistically significant segment comprises:
determining a segment score for the segment based on a segment sequence read count for the segment; and
determining a segment variance estimate for the segment,
wherein determining whether the segment is a statistically significant segment is based on the segment score and the segment variance estimate for the segment.

31. A method comprising:
obtaining a first sequence read from a first sample and a second, corresponding sequence read from a second sample, the first sequence read and the second sequence read categorized in at least one bin of a plurality of bins of a genome;
determining that a first bin in which the first sequence read is categorized and a corresponding second bin in which the second sequence read is categorized are statistically significant based on a number of sequence reads that are categorized in the first bin and the second bin, respectively, and a bin variance estimate for the first bin and the second bin, respectively;
determining that a first segment of the genome corresponding to the first sample and a second segment of the genome corresponding to the second sample are statistically significant based on a number of sequence reads that are categorized in bins included in the first segment and second segment, respectively, and based on a segment variance estimate of the first segment and second segment, respectively; and
identifying a source of a copy number change in the first sample indicated by the first bin and the first segment based on a comparison of the first to the second bin and a comparison of the first segment to the second segment.
